(19) United States
(12) Patent Application Publication
(10) Pub. No.: US 2025/0372227 A1
(43) Pub. Date: Dec. 4, 2025

(54) MEDICANT DELIVERY SYSTEM, METHODS OF PROVIDING RECOMMENDATIONS REGARDING MEDICANT DELIVERY, AND RELATED SYSTEMS AND METHODS

(71) Applicant: Insulet Corporation, Acton, MA (US)

(72) Inventors: Joon Bok Lee, Acton, MA (US); Ashutosh Zade, San Diego, CA (US); Yibin Zheng, Hartland, WI (US); Jason O'Connor, Acton, MA (US)

(21) Appl. No.: 19/222,755

(22) Filed: May 29, 2025

Related U.S. Application Data
(60) Provisional application No. 63/654,659, filed on May 31, 2024.

(51) Int. Cl. G16H 20/17; A61M 5/142; G16H 40/63

(52) U.S. Cl. CPC G16H 20/17; A61M 5/14248; G16H 40/63; A61M 2005/14208; A61M 2005/14292; A61M 2202/0007; A61M 2202/04; A61M 2205/3303; A61M 2205/50

(57) ABSTRACT

A system for administration of medicament to a user-body includes an analyte sensor and an automated medicament delivery device. The automated medicament delivery device is configured to determine an actual bolus fraction of a total daily medicament that was delivered as a total daily bolus dose, determine a ratio of the actual bolus fraction relative to a target bolus fraction, and based at least partially on the determined ratio of the actual bolus fraction relative to a target bolus fraction, determine at least one new value of a parameter value utilized by the automated medicament delivery device to determine bolus doses.

### CROSS-REFERENCE TO RELATED APPLICATIONS

This application claims the benefit under 35 U.S.C. § 119(e) of U.S. Provisional Patent Application Ser. No. 63/654,659, filed May 31, 2024, the disclosure of which is hereby incorporated herein in its entirety by this reference.

### TECHNICAL FIELD

The present disclosure generally relates to medicament delivery systems. More particularly, the present disclosure relates to automated medicament delivery algorithms and adjusting parameters utilized in determining bolus dosing.

### BACKGROUND

Automated medicament delivery devices (“AMD,” e.g., an Automated Insulin Delivery (AID) device, without limitation) are often used to administer medicaments to the body of a patient via a cannula inserted into the body to treat medical conditions (e.g., Type 1 Diabetes, without limitation).

A bolus of medicament (e.g., a correction bolus or a carbohydrate bolus (also called a “meal bolus,”) without limitation) may be delivered by the AMD to the user-body as an immediate bolus (a specified amount of medicament administered in a single dose), an extended bolus (a specified amount of medicament administered as a sequence of discrete doses at a constant rate over a set duration of time), or a combination bolus (a portion of a specified amount of medicament administered immediately in a single dose and the remainder administered over a set duration of time).

### BRIEF SUMMARY

In one or more examples, a system for administration of medicament to a user-body may include an analyte sensor and an automated medicament delivery device. The automated medicament delivery device may include at least one processor and at least one non-transitory computer-readable storage medium storing instructions thereon that, when executed by the at least one processor, cause the automated medicament delivery device to: determine an actual bolus fraction of a total daily insulin that was delivered as a total daily bolus dose, determine a ratio of the actual bolus fraction relative to a target bolus fraction, and based at least partially on the determined ratio of the actual bolus fraction relative to a target bolus fraction, determine at least one new value of a parameter utilized by the automated medicament delivery device to determine bolus doses.

In one or more examples, a method for recommending new values of parameters for determining bolus doses may include determining an actual amount of a total daily insulin that was delivered as bolus doses, determining a ratio of the actual amount relative to a target amount, and based at least partially on the determined ratio, determining at least one new value of a parameter to utilize in calculating bolus doses.

In one or more examples, a system for administration of medicament to a user-body may include an analyte sensor and an automated medicament delivery device. The automated medicament delivery device may include at least one processor and at least one non-transitory computer-readable storage medium storing instructions thereon that, when executed by the at least one processor, cause the automated medicament delivery device to: determine insulin deviations made by the automated medicament delivery device subsequent to administration of bolus dose during postprandial periods throughout a given period of time and based at least partially on the determined insulin deviations, determine at least one new value of a parameter to utilize in determining bolus doses.

Other technical features may be readily apparent to one skilled in the art from the following figures, descriptions, and claims.

Within the scope of this application, it should be understood that the various aspects, embodiments, examples and alternatives set out herein, and individual features thereof may be taken independently or in any possible and compatible combination. Where features are described with reference to a single aspect or embodiment, it should be understood that such features are applicable to all aspects and embodiments unless otherwise stated or where such features are incompatible.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a schematic diagram illustrating a medicament delivery system in accordance with one or more examples;

FIG. 2 is a block diagram of a medicament delivery system for controlled administration of medicament in accordance with one or more examples;

FIG. 3 shows a flowchart of method of determining recommendations for delivering medicant to a user according to one or more embodiments of the present disclosure;

FIG. 4 shows a flowchart of method of determining recommendations for delivering medicant to a user according to one or more embodiments of the present disclosure; and

FIG. 5 shows a flowchart of method of determining recommendations for delivering medicant to a user according to one or more embodiments of the present disclosure.

### DETAILED DESCRIPTION

While the specification concludes with claims particularly pointing out and distinctly claiming what are regarded as embodiments of the present disclosure, various features and advantages may be more readily ascertained from the following description of example embodiments when read in conjunction with the accompanying drawings, in which:

Illustrations presented herein are not meant to be actual views of any particular automated medicament delivery device, insulin pump, component, or system, but are merely idealized representations that are employed to describe embodiments of the disclosure. Additionally, elements common between figures may retain the same numerical designation for convenience and clarity.

The following description provides specific details of embodiments. However, a person of ordinary skill in the art will understand that the embodiments of the disclosure may be practiced without employing many such specific details. Indeed, the embodiments of the disclosure may be practiced in conjunction with conventional techniques employed in the industry. In addition, the description provided below does not include all the elements that form a complete structure or assembly. Only those process acts and structures necessary to understand the embodiments of the disclosure are described in detail below. Additional conventional acts and structures may be used. The drawings accompanying the application are for illustrative purposes only, and are thus not drawn to scale.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structures and the apparatus in a predetermined way.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.).

As used herein, the term “and/or” includes any and all combinations of one or more of the associated listed items.

Manual insulin deliveries are often utilized in conjunction with automated insulin delivery (AID) in insulin pumps with AID algorithms for controlling operation of the insulin pumps to maximize the glucose control outcomes of insulin pump users. However, while such AID systems and/or algorithms are typically designed to compensate for a wide range of variations in user bolus dosing behaviors, the AID algorithms are often specifically tailored to yield the most “time in range” (e.g., a percentage of time that a user's blood glucose levels remain in a specific target range) if the user's behaviors follow specific patterns that better compensate for the user's individual disturbances in glucose concentrations. Accordingly, if a user's behavior does not follow those specific patterns, the user may experience sub-optimal glucose control outcomes.

Embodiments of the present disclosure include systems and methods for assessing a total insulin delivery that is delivered by AID algorithms versus a user's manual insulin delivery patterns and recommending potential changes in the user's clinical parameters for manual insulin delivery (e.g., bolus dosing) such that the total insulin delivery by the user's manual boluses may better match optimal insulin delivery patterns for the user's individual glucose disturbances.

The clinical parameters (e.g., an insulin to carb ratio and/or a correction factor) may be directly modified based on differences in proportion of actual manual insulin delivery versus a target manual insulin delivery. For example, the clinical parameters may be adjusted by a ratio between a target proportion of manual bolus delivery (such as 0.5) of the total insulin delivery versus an actual proportion of the total insulin delivery. For instance, if the user's actual proportion of manual insulin delivery is 0.3 or 30%, rather than 0.5 or 50%, the clinical parameters would be multiplied by 0.3/0.5, or 60%. As a result, if the user repeats a same bolus request in a subsequent insulin pump session, the manual insulin delivery (e.g., a user-instructed bolus dose) would be increased, and the total manual insulin delivery will more closely match the target proportion of manual bolus delivery.

As a non-limiting example, if the total manual insulin delivery within a previous day (e.g., 288 cycles) were 10 units, with a total insulin delivery of 24 units, the user's proportion of manual insulin delivery to total insulin delivery would be 0.41 (i.e., 10/41). In comparison to a typical basal/bolus split ratio of 0.5, the systems and methods of the present disclosure above may recommend the clinical parameters be decreased by, for example, multiplying the clinical parameters by 0.41/0.5 or 0.82. Exemplary clinical parameters that may be adjusted include an insulin to carb ratio and/or a correction factor. The decrease in the clinical parameters may result in an increase in manual insulin delivery if the same carbohydrate ingestion and/or a same glucose excursion is requested to be compensated by a bolus request.

In additional embodiments, the clinical parameters can be adjusted by considering an actual extent of under- or over-delivery that needed to be executed by an AID algorithm following each user bolus dose and adjusting the clinical parameters based on extent to which the user's blood glucose levels needed to be corrected subsequent to bolus doses. Specifically, insulin delivery excursions following every meal bolus may be assessed to determine whether the user is under- or over-delivering at each manual or user-requested insulin delivery. The amount of insulin delivered and/or suspended to correct the user's blood glucose levels subsequent to bolus doses may be utilized to adjust the clinical parameters.

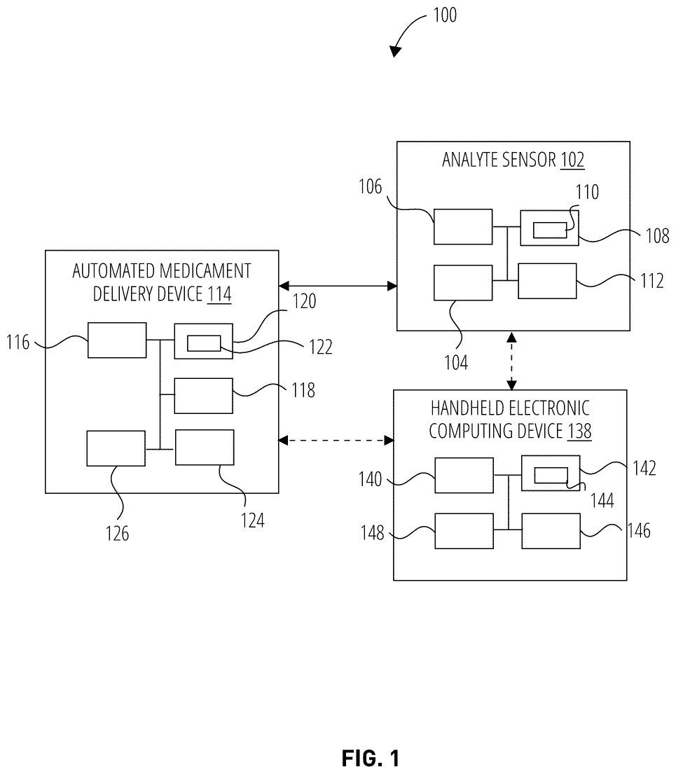

FIG. 1 is a schematic diagram showing a system 100 for administration of medicament to a user-body, in accordance with one or more examples.

In one or more examples, the system 100 may be capable of one or more modes of operation of administration of medicament (e.g., one or more distinct modes of operation, without limitation). Non-limiting examples of the one or more modes of operation include: fully automated administration of medicament, partially automated administration of medicament, and/or manual administration of medicament. In one or more examples, the system 100 may be capable of alternating between the multiple (e.g., two or more, without limitation) modes of operation. As a non-limiting example, the system 100 may alternate between one or more of: fully automated operation, partially automated operation, and/or manual operation.

The system 100 may administer medicament at least partially based on one or more values representative of amounts of one or more analytes present within a user-body (such values respectively an “analyte value”). The one or more analytes may include constituents of the user-body and foreign substances, such as medicaments, markers, metabolites, and combinations or sub combinations thereof, without limitation. Analyte values may include values representative of amounts of one or more analytes present within a user-body and values at least partially based on the same, such as, an A1C value, a blood glucose value (e.g., milligrams per decaliter (mg/dL), without limitation), an insulin-to-carbohydrate (I:C) ratio, or any combination thereof, without limitation.

The system 100 may also administer an amount of medicament at least partially based on user inputs (e.g., a user defined bolus amount or details related to a meal consumed or about to be consumed, such as number of carbohydrates, amount of fat, and amount of protein, without limitation). As used herein, administration of medicament responsive to a user input may be referred to as manual medicament delivery or manual delivery.

Non-limiting examples of medicaments administrable by system 100 include: insulin, glucagon-like peptide-1 receptor agonist (GLP-1), glucose-dependent insulinotropic polypeptide (GIP), or other hormones, insulin substitutes, and combinations of medicaments, such as two or more of insulin, GLP-1, and GIP, or other like hormones. While specific examples discussed herein may involve insulin or GLP-1, or GIP, this disclosure is not limited to those examples, and other medicaments do not exceed the scope. As a non-limiting example, glucagon, morphine, analgesics, fertility medicaments, blood pressure medicaments, chemotherapy drugs, arthritis drugs, weight loss drugs, without limitation are non-limiting examples of medicaments that are specifically contemplated. system 100.

The system 100 includes an analyte sensor 102 and an automated medicament delivery device 114. The system 100 may optionally include a handheld electronic computing device 138.

The analyte sensor 102 may be configured to obtain data related to one or more analytes within the user-body (“analyte data”). In various examples, the analyte data may include one or more analyte values. In various examples, the analyte sensor 102 is an analytical bio-sensing device, such as a continuous glucose monitor (CGM) or an integrated continuous glucose monitor (iCGM) (e.g., examples of commercially available analytical bio-sensing devices include the FREESTYLE LIBRE® 3 manufactured by Abbott or the DEXCOM® G6 manufactured by Dexcom, without limitation).

The analyte sensor 102 may include a filament 104 and various electronic components. The filament 104 may be configured to obtain data related to one or more analytes within a user-body and provide the data to the various electronic components of the analyte sensor 102. The filament 104 may be configured to obtain the data directly from fluids of a user-body, including without limitation interstitial fluids of a user-body, from tissue of a user-body, combinations thereof, or in any other manner known in the art.

The analyte sensor 102 may include one or more processors 106, a memory 108, and communication equipment 112. The memory 108 may be coupled to the one or more processors 106. The memory 108 may be used for storing data, metadata, and programs for execution by the one or more processors 106. The memory 108 may include storage for storing data or instructions 110. The instructions 110 may include instructions for processing data obtained via the filament 104. When the instructions 110 are executed by the one or more processors 106, the instructions 110 cause the one or more processors 106 to process the data obtained via the filament 104. The instructions 122 may be implemented in hardware (e.g., one or more hardware processors of the one or more processors 106, such as an integrated circuit, application specific integrated circuit (ASIC), digital signal processor (DSP), or other logic circuit, without limitation), implemented in software (e.g., firmware, software, machine code, applications, without limitation), or a combination thereof. The instructions 110 for processing the data obtained via the filament 104 may include one or more instructions respectively for determining analyte values at least partially based on the data, or for sending the data, analyte values or both to the automated medicament delivery device 114 and/or the handheld electronic computing device 138.

The communication equipment 112 is configured to facilitate communication (e.g., a device or interface for wired communication, wireless communication, both wired and wireless communication, without limitation) of the analyte sensor 102 with other devices, including the automated medicament delivery device 114 and/or the handheld electronic computing device 138, without limitation. Such communication may be according to any appropriate wired or wireless communication protocol, such as WI-FI®, BLUETOOTH®, near-field communication (NFC), radio-frequency identification (RFID), or any other radio-frequency, infrared, or optical communication technology.

The automated medicament delivery device 114 may be configured to administer medicament to a user-body, such as subcutaneously into the user-body, without limitation, in accordance with one or more examples. In one or more examples, the automated medicament delivery device 114 may offer one or more modes of operation for administration of medicament to a user-body. When operating in some of the modes of operation, the automated medicament delivery device 114 may administer medicament at least partially responsive to analyte values, including without limitation analyte values received from analyte sensor 102. When operating in some further modes of operation, the automated medicament delivery device 114 may administer medicament at least partially responsive to user input. When operating some yet further modes of operation, the automated medicament delivery device 114 may administer medicament at least partially responsive to both analyte values and user input. Non-limiting examples of the one or more modes of operation offered by the automated medicament delivery device 114 include: fully automated administration of medicament, partially automated administration of medicament, or manual administration of medicament.

When operating in an operative mode that includes manual administration of medicament, the automated medicament delivery device 114 may administer medicament solely in response to a user input (e.g., delivers medicament in response to a user confirmation of delivery of medicament or in response to a user instruction to delivery medicament, without limitation). When operating in an operative mode that includes fully automated administration of medicament, the automated medicament delivery device 114 may administer medicament solely in response to analyte values (e.g., delivers medicament in response to one or more analyte values, without limitation). When operating in an operative mode that includes partially automated administration of medicament, the automated medicament delivery device 114 may administer medicament in response to analyte values and user input (e.g., delivers medicament in response to a user input and an analyte value, or alternately delivers medicament in response to a user input or in response to analyte values, without limitation).

Medicament administration may include administration of a basal amount of medicament regularly delivered over one or more control intervals (e.g., at one or more determined basal rates during different periods of the day, without limitation) to keep analyte levels stable and within a determined or predetermined range. Medicament administration may also include administration of bolus amounts of medicament administered as an immediate bolus, an extended bolus, or a combination bolus (combination of an immediate bolus and an extended bolus). The bolus amount of medicament may be a correction bolus responsive to a change in analyte levels or a user defined bolus (e.g., responsive to user inputs provided, such as a user defined bolus amount or details related to a meal consumed or about to be consumed, such as number of carbohydrates, amount of fat, and/or amount of protein, without limitation).

The automated medicament delivery device 114 may include a delivery system 116, a power source 126, one or more processors 118, a memory 120, and communication equipment 124. In one or more examples, the automated medicament delivery device 114, or portions thereof, may include a wearable device and may be secured to a user-body (e.g., secured via one or more adhesive layers attaching the automated medicament delivery device 114 to the skin of the user-body or a material that is secured to the user body, without limitation).

In various examples, the delivery system 116 is configured to cause an amount of medicament to move (e.g., flow, without limitation) toward and/or into a user-body.

In various examples, delivery system 116 may deliver amounts of medicament at least partially responsive to requests. In various examples, instructions 122 of the memory 120 may include instructions for determining and generating requests for delivery system 116. In various examples, instructions 122 may include instructions for determining one or more amounts of medicament, determining a timing for delivery of one or more amounts of medicament, and for generating one or more requests for delivery system 116 related to the same. When such instructions of instructions 122 are executed by one or more processors 118, the one or more processors 118 determine the amounts of medicament and timing of delivery, generate requests for the delivery system 116 at least partially based on the determined amounts and timing, and provide the requests to delivery system 116. In some embodiments, the requests and/or instructions for generating requests may be received from the handheld electronic computing device 138. Furthermore, activity (e.g., determined and generated requests for delivery system 116, administered doses, etc.) of the automated medicament delivery device 114 may be stored in the memory 120.

The communication equipment 124 is configured to facilitate communication (e.g., wireless communication, without limitation) of the automated medicament delivery device 114 with other devices, including, without limitation, communication between analyte sensor 102 and the automated medicament delivery device 114 and communication between the automated medicament delivery device 114 and the handheld electronic computing device 138. The communication may be wired or wireless communication and may utilize any suitable communication protocol such as wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In various examples, the communication equipment 124 includes an Internet of Things (IOT) Subscriber Identity Module (SIM) card (e.g., a machine-to-machine SIM card, a Universal Integrated Circuit Card, without limitation).

The power source 126 is configured to supply power to the delivery system 116 and the various electronic components, such as the one or more processors 118, memory 120, communication equipment 124, etc. The power source 126 may be, as a non-limiting example, a power storage device (e.g., a battery, without limitation), a power inlet, a power regulator, or combination thereof.

In various examples, the handheld electronic computing device 138 is configured to communicate with the automated medicament delivery device 114 and the analyte sensor 102. The handheld electronic computing device 138 may be chosen from among a dedicated electronic device, a smart phone, a tablet computer, a wearable device (e.g., a smart watch, without limitation), a cloud computing device, and the like.

The handheld electronic computing device 138 may include one or more processors 140, memory 142 that stores instructions 144 to be executed by the one or more processors 140, communication equipment 146, and a user interface 148. The one or more processors 140 and memory 142 may be configured/programmed to perform any of the operations discussed above, as well as other control operations for managing the automated medicament delivery device 114 and the analyte sensor 102.

The communication equipment 146 is configured to facilitate communication (e.g., wireless communication, without limitation) of the handheld electronic computing devices 138 with other devices, such as the automated medicament delivery device 114 and the analyte sensor 102. The communication may be wired or wireless communication, such as via a wireless networking protocol (e.g., Wi-Fi®, without limitation), a short-range wireless protocol (e.g., BLUETOOTH®, without limitation), a near-field communication standard, a cellular standard, or any other wireless optical or radio-frequency protocol. In some of these examples, the automated medicament delivery device 114 and the handheld electronic computing devices 138 are paired via the short-range wireless protocol (e.g., paired via BLUETOOTH®, without limitation) and successful message transmissions between the automated medicament delivery device 114 and the handheld electronic computing devices 138 may be acknowledged.

The user interface 148 is configured to provide a user with information and obtain information from the user via one or more of a display, an audio speaker, an LED, a vibration motor, a button (e.g., a mechanical button, capacitive button, without limitation), a gesture-based interface, and the like.

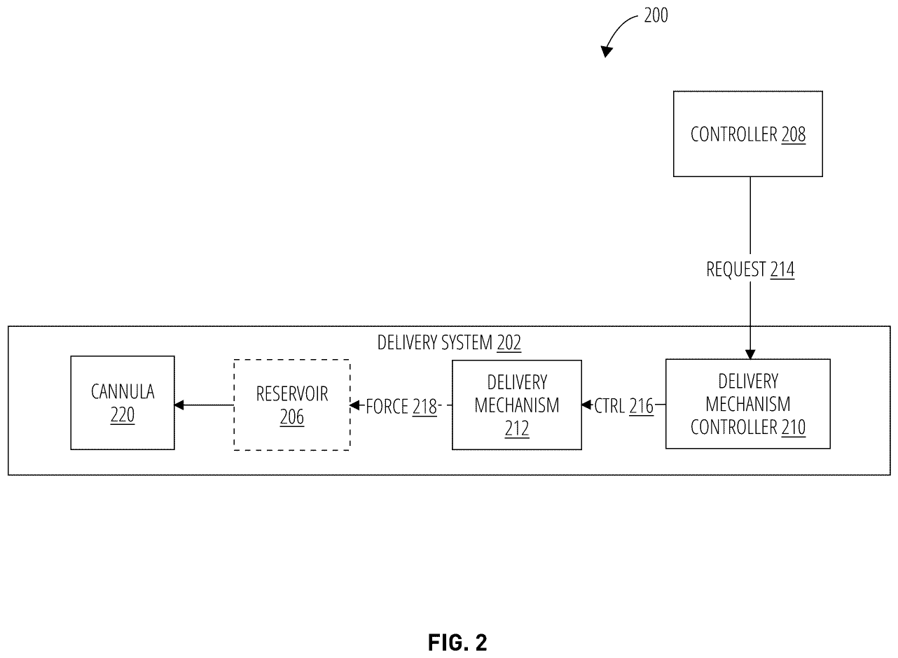

FIG. 2 is a block diagram of a medicament delivery system 200 for controlled administration of medicament to a user-body, in accordance with one or more examples.

The controller 208 is configured to manage the automated medicament delivery device 114 and, more generally, administration of medicament to a user-body. In one or more examples, controller 208 may be implemented by instructions 122 and one or more processors 118 of automated medicament delivery device 114 of FIG. 1. Furthermore, activity (e.g., determined and generated requests for delivery system 116, administered doses, etc.) of the automated medicament delivery device 114 and/or the medicament delivery system 200 may be stored in a memory.

In various examples, the controller 208 and the delivery system 202 may be realized in different devices (e.g., controller 208 may be realized in a physically different device (or devices) than delivery system 202 is realized, such as the handheld electronic computing device 138, without limitation), or in the same device. When realized in different devices, functionality of the controller 208 and the delivery system 202 may be implemented, at least in part, by respective memory and one or more processors of their respective devices. When realized in a same device, functionality of the controller 208 and the delivery system 202 may be implemented, at least in part, by like memory and like one or more processors, respective memory and respective one or more processors, or a combination thereof. Non-limiting examples of devices in which the controller 208, or a portion thereof, may be realized include: a handheld electronic computing device, such as a dedicated electronic device, a smart phone, a tablet computer, a wearable device (e.g., a smart watch, without limitation), a cloud computing device, and the like.

In various examples, the controller 208 may be configured to receive analyte data (e.g., from the analyte sensor 102, without limitation) including analyte values. In one or more examples, the controller 208 may determine information about analytes within a user-body at least partially based on analyte data, for example, amounts, trends, distributions, without limitation. The controller 208 may analyze information about analytes in a user-body and may present the information and/or analysis to a patient, caregiver, or healthcare provider, as a non-limiting example, via an application (e.g., executing on a personal computer, a smart phone, a cloud server, and/or combinations thereof).

In various examples, the controller 208 may be configured to receive information from inputs from the patient or a caregiver (e.g., when the patient is going to eat or ate a meal, the size of the meal or the number of carbohydrates, when the patient exercised or plans to exercise, the duration and/or intensity of the exercise, etc., without limitation), and inputs from other electronic devices (e.g., information from a smartwatch, without limitation) and to utilize such information as discussed herein. For example, in various examples, the controller 208 may utilize some or a totality of such information to determine amounts of medicament to administer and timing of administration of medicament. Further, controller 208 may also be configured to determine requests, including requests 214 to administer doses, and send those requests to the automated medicament delivery device 114.

In various examples, the controller 208 may be configured to determine a target dose amount to administer to a user of the medicament delivery system 200. The controller 208 may determine a target dose amount at least partially based on therapy or clinical parameters, meal information, analyte values, and a control algorithm, without limitation.

In the context of insulin therapy to treat diabetes, therapy or clinical parameters may include insulin sensitivity factor (ISF), carbohydrate ratio (CR), amount of daily dose of long-acting insulin (LAI), a current glucose value, and derivatives thereof without limitation. The timing, duration of delivery, and target dose amounts associated with requests generated by controller 208 may be governed by one or more control algorithms, discussed below. For instance, the timing, duration of delivery, and target dose amounts associated with requests generated by controller 208 may be governed by one or more AID algorithms.

The controller 208 may send a request 214 to administer a dose to delivery system 202, and more specifically, delivery mechanism controller 210. The request 214 to administer a dose may include the target dose amount determined by the controller 208.

The cannula 220 is insertable into a user-body (e.g., with a tip thereof positioned subcutaneously, without limitation) and is configured to provide medicament to a user-body (e.g., subcutaneously into the user-body, without limitation).

The reservoir 206 is configured to store and retain a medicament therein. As a non-limiting example, the reservoir 206 may be a hollow body, a chamber, or a vial, without limitation. In various examples, the reservoir 206 is a fluid reservoir for holding medicament and may be, as a non-limiting example, formed from the walls of a cartridge. In the cartridge example, the delivery system 202 may include a chamber (i.e., a space or region defined within the delivery system 202) configured to receive and hold a prefilled (prefilled with medicament) cartridge, eject an exhausted cartridge, and optionally receive a prefilled cartridge to replace (i.e., a replacement cartridge) the exhausted cartridge. Generally speaking, a volume of fluid in the reservoir 206 will be greater in a pre-filled state than the volume in an exhausted state. Additionally, or alternatively to the cartridge example, the delivery system 202 is a multi-part delivery device where one of the two parts includes the reservoir 206 and the other one of the two parts includes the delivery mechanism controller 210. The other one of the two parts may optionally further include the controller 208. Either one of the two parts may optionally include the delivery mechanism 212 (e.g., a pump mechanism, without limitation). The one of the two parts that includes the reservoir 206 is disposable (i.e., a “disposable part”) and configured to be removably secured to the other part of the medicament delivery system 200. When the reservoir 206 is exhausted, the disposable part may be removed and a replacement part including a reservoir 206 or a cartridge, optionally in a pre-filled state, may be removably secured to the other part of the medicament delivery system 200.

The delivery mechanism 212 is configured to urge fluid in the reservoir 206 toward an interface for dispensing fluid (interface not shown). In various examples, the delivery mechanism 212 may be positioned adjacent to the reservoir 206. The delivery mechanism 212 is configured to cause an amount of the medicament to be administered to the user-body by causing the amount to flow from the reservoir 206 toward and into a user-body via cannula 220, which is in fluidic communication with the reservoir 206. In various examples, the delivery mechanism 212 may utilize any suitable mechanism to generate positive displacement or negative displacement to transfer amounts of medicament from the reservoir 206 toward the cannula 220 and a user-body. Non-limiting examples of mechanisms include a ratchet gear pump, a peristaltic pump, a linear peristaltic pump, a piston pump, a gear pump, a reciprocating pump, a diaphragm pump, a bellows pump, or a diaphragm pump.

For example, the delivery mechanism 212 may apply a force to an urging mechanism (e.g., a plunger, flexible-walled tube, a diaphragm, without limitation) free to move within the reservoir 206 or within a fluid line of delivery mechanism 212, and via such a force, move the fluid directly, or the urging mechanism in a direction that urges fluid in the reservoir 206 toward the aforementioned interface. In one or more examples, the delivery mechanism 212 may include an electrical motor (e.g., an AC or DC motor) that produces a force to, directly or indirectly, move the urging mechanism to perform a delivery action. A delivery action dispenses at a predetermined rate (i.e., a predictable amount of fluid over a predictable duration of time). The delivery mechanism 212 may be capable of multiple rates of delivery, and in one or more examples, may be preconfigured to use a same rate of delivery all the time, or, in some cases, may be provided discretion to determine a rate of delivery consistent with a target dose amount included with a request 214.

Such an electric motor may be a current controlled electric motor, voltage controlled electric motor, pulse-width controlled electric motor, or combination or sub combination thereof. Such an electronic motor may be directly or indirectly digitally controlled. A control signal 216 may be determined and generated by the delivery mechanism controller 210 to correspond to a delivery action. The control signal 216 may also be referred to herein as a “command 216” or an “instruction 216.”

The delivery mechanism controller 210 may generate control signals 216 corresponding to one or more delivery actions at least partially based on a request 214 to administer a dose received from the controller 208 or elsewhere (e.g., the handheld electronic computing device 138). The control signal 216 may include first control signals to cause the delivery mechanism 212 to generate a resultant force 218, and a second, different control signal to cause drive delivery mechanism 212 to not or stop generating the force 218. Utilizing control signals 216, the delivery mechanism controller 210 may control a length of a duration of time that the delivery mechanism 212 produces the force 218 and applies it to dispense fluid from the reservoir 206, and indirectly, an amount of fluid dispensed from the reservoir 206.

When delivery mechanism controller 210 generates the control signal 216 in response to a request 214 to administer a dose from the controller 208 or elsewhere, it may generate the control signal 216 at least partially based on a value of a target dose amount included with, or indicated by, the request 214 to administer a dose. One or more delivery actions may be utilized to dispense an amount fluid corresponding to a dose amount determined by the controller 208. For example, a fluid amount dispensed according to a delivery action may be less than a dose amount. Generally speaking, the delivery mechanism 212 and the delivery system 202 are agnostic to the purpose for which fluid is dispensed and unaware of what constitutes a working amount of fluid to administer a dose, or series of doses, of medicament. So, while it may be desirable that a fluid amount dispensed according to one or more delivery actions will be exactly the same as a target dose amount, some negligible difference is specifically contemplated, and what is considered “negligible” will depend on specific operation conditions.

In one or more examples, the delivery mechanism controller 210 may be configured to determine and generate feedback information about delivery actions, such as times of delivery actions and dispensed amounts, without limitation. Feedback information may be generated based on information generated by the delivery mechanism 212 or by sensors utilized by the delivery mechanism controller 210 to monitor operation of the delivery mechanism 212 (sensors not depicted). For example, sensors to monitor mechanical movement, current consumption, a voltage profile of an electric motor, without limitation. Such information may be logged and provided to and stored at the controller 208 and/or the handheld electronic computing device 138, without limitation, for later processing or reading, without limitation. For example, the logs can be processed to determine patterns that may be utilized to determine whether the delivery system 202 is operating as expected (e.g., in a predictable manner, without limitation), and if a difference between actual and expected operation exceeds a threshold, the delivery mechanism controller 210 may be updated (e.g., firmware, parameters, or both, of the delivery mechanism controller 210 may be updated, without limitation) to compensate or correct for the difference. Additionally or alternatively to updating the firmware or parameters, in a multi-part system, one or more parts including the delivery mechanism controller 210 or the delivery mechanism controller 210 may be indicated as needing replacement (e.g., an alarm or alert is generated at the delivery system 202, the medicament delivery system 200, a mobile device, and/or computer in communication therewith, without limitation).

### Control Algorithm Architecture

As noted above, values of target dose amounts and timing of requests to administer the target dose amounts generated by the controller 208 may be governed by one or more control algorithms (AID or AMD algorithms) implemented at the controller 208. Generally speaking, such a control algorithm may, via one or more control actions, try to cause an amount of analyte in the body (represented by values captured by, or at least partially based on, an analyte sensor or monitor, without limitation) to track (e.g., at least substantially match) a target amount or range of analyte (in control terms, the target amount of analyte is the “set point”) in the body. The control actions may include an amount, a timing, and a duration of administration of doses of medicament that functions as a therapeutic agent in the body.

In one or more examples, a control algorithm may employ a modular design in which core functionality may be separated from dependent functionality. Dependent functionality includes, as non-limiting examples, functionality that may be implementation-specific to a current environment, such as software abstraction for an analyte sensor. Such dependent functionality may include software services which interface with implementation-specific features that affect inputs or outputs to the control algorithm. Dependent functionality may include, as a non-limiting example, functionality for managing algorithm initialization and upload of administration history, managing the control algorithm's state and data variables, and maintaining cycle-to-cycle data utilized by the algorithm such as analyte values, current or historical. Dependent functionality may include functionality responsible for sending requests to administer doses to delivery system 202 which are determined by the control algorithm.

Transmission of data, including without limitation, a request 214 to administer a dose, may occur over wired, wireless, or a combination thereof communication paths, in a synchronous or asynchronous manner. In one or more examples, the control algorithm may include one or more layers to provide safety or other operational constraints (e.g., for edge case handling, without limitation).

In one or more examples, a control algorithm may determine a target dose amount to include within a request at least partially based on a dynamic model of a user-body's response, in terms of amount of analyte in the user-body, to administration of analyte to the user-body. The control algorithm may determine a future amount of analyte or a change in amount of analyte over a predetermined duration of time for a respective dose amount and compare the determined future amount or change to a target amount or change. The control algorithm may determine target dose amounts according to control intervals that occur according to a predetermined schedule, on-demand, or both. In one or more examples, the control intervals may correspond to diurnal intervals such as day-night, weeks, days, twenty-four (24) hours, single hours, and sub-intervals of the same, such as 5-minute intervals.

In some cases, the control algorithm may be or include a control algorithm that handles constraints, such as a model-predictive-control (MPC) algorithm. Non-limiting examples of constraints include: upper and lower bounds on analyte levels that can be set to prevent dangerous hypo- or hyperglycemia; medicament delivery rates capable by delivery system 202 can be constrained to prevent over- or under-dosing; and considerations related to medicament-on-board to, e.g., prevent stacking of medicament doses waiting to work on analyte in the body (e.g., stacking of insulin waiting to work on glucose, without limitation).

One or more examples discussed herein may refer to administering medicament or a medicament therapy to a user or the user-body. Such discussion is intended to encompass examples where medicament or a medicament therapy is administered to a user by automated medicament delivery devices discussed herein, examples where requests to administer doses in accordance with administering medicament or medicament therapy to the user or user-body are generated by a controller and sent to a delivery device, and examples where instructions (e.g., control signals, without limitation) in accordance with dose amounts and timing included with such requests to administer doses are generated by a delivery mechanism controller and sent to a delivery mechanism.

In some embodiments, the automated medicament delivery device 114 and an AID algorithm governing one or more operations of the automated medicament delivery device 114 may be optimized (e.g., tuned) to incorporate a user's typical insulin needs based on an assumption that 50% of the user's insulin needs are fulfilled by manual insulin delivery responsive to the user eating meals (i.e., bolus doses). The foregoing assumption can be represented by Equation 1.

$${B}_{AID} ( i ) = \frac{0.5 \cdot TDI ( i )}{24} \qquad Equation 1$$

with B_{AID} representing the user's insulin needs in terms of basal insulin delivery per hour for the i^{th} automated medicament delivery device 114. Accordingly, the B_{AID} is calculated by dividing 50% of the user's TDI by twenty-four (24) to convert the amount of insulin to deliver into hourly increments. In some embodiments, the TDI may be determined based on a user's insulin delivery history, which may be stored in one or more of the memory 120 of the automated medicament delivery device 114, a memory 142 of the handheld electronic computing device 138, a remote device (e.g., a server), or any other location.

However, as mentioned briefly above, a user's manual insulin delivery patterns may vary from the assumption that 50% of the user's TDI is delivered via bolus doses. In other words, an amount of insulin delivered via the user's manual insulin delivery (i.e., the percentage of TDI that is delivered as manual insulin delivery) during a day may vary from a target amount. Accordingly, bringing a user's manual insulin delivery patterns into alignment with the above assumption helps to optimize the effectiveness of the automated medicament delivery device 114 and the AID algorithm in maintaining a user's blood glucose levels within a desired range. The 50% value throughout this embodiment is merely an example, and this assumption may vary depending on the value of this optimal manual insulin delivery pattern that results in best performance by the automated medicament delivery device.

In some embodiments, the automated medicament delivery device 114 and the AID algorithm governing one or more operations of the automated medicament delivery device 114 are configured for basal dosing only, and manual doses and correction doses are administered via other devices. In other embodiments, the automated medicament delivery device 114 and the AID algorithm are configured to administer basal dosing in addition to other dose types, such as bolus doses, correction doses, etc.

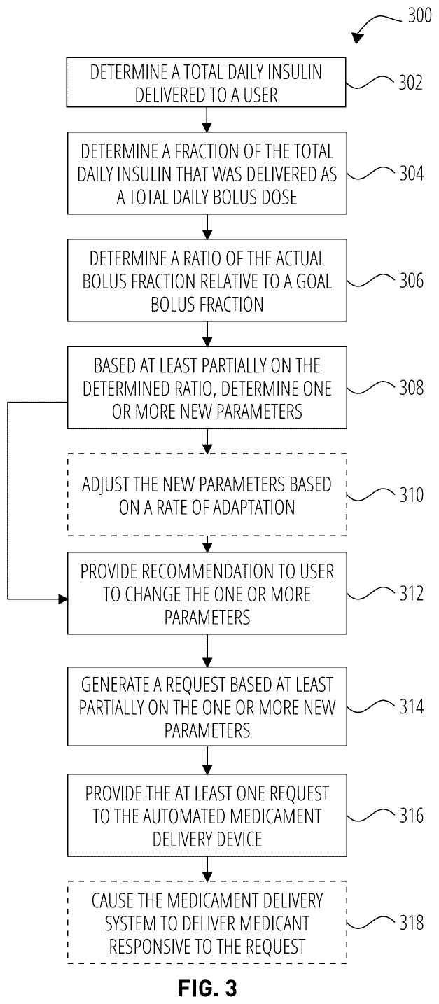

FIG. 3 is a flowchart of a method 300 of recommending new clinical parameters to be utilized to calculate manual insulin doses (i.e., bolus doses) according to one or more embodiments of the present disclosure. In particular, the method 300 may include recommending new clinical parameters utilized to calculate bolus doses based at least partially on a proportion of a total actual amount of insulin delivered manually (referred to hereinafter as “total daily bolus dose”) during a given 24-hr period relative to a target total amount of insulin to be delivered manually (referred to hereinafter as “target daily bolus dose”) during the given 24-hr period. For purposes of the present disclosure, the terms “manual dose,” “manual insulin dose,” and “bolus dose” and derivatives of those terms are used interchangeably to refer to a “bolus dose.” In some embodiments, one or more acts of the method 300 may be performed and/or executed by the controller 208 of the medicament delivery system 200. However, the disclosure is not so limited, and one or more acts of the method 300 may be performed, executed, and/or initiated by the delivery mechanism controller 210 of the delivery system 202, other controllers of the system 100, the handheld electronic computing device 138, and/or one or more other remote devices. For purposes of the present disclosure, the acts of the method 300 are described as being performed, executed, and/or initiated by the controller 208 of the medicament delivery system 200.

In some embodiments, the method 300 may include determining a total daily insulin (“TDI”) delivered to a user-body during a given 24-hr period (e.g., a day), as shown in act 302 of FIG. 3. For example, the controller 208 of the medicament delivery system 200 may determine the TDI delivered to a user-body during a given 24-hr period based at least partially on the generated requests 214 to administer doses and/or recorded activity of the medicament delivery system 200 and/or automated medicament delivery device 114 during the 24-hr period. The TDI includes basal doses automatically delivered to the user-body via the automated medicament delivery device 114 according to an AID algorithm during the 24-hr period and all bolus doses administered during the 24-hr period. As noted above, in some embodiments, the TDI may be determined based on a user's insulin delivery history, which may be stored in one or more of the memory 120 of the automated medicament delivery device 114, a memory 142 of the handheld electronic computing device 138, a remote device (e.g., a server), or any other location.

Additionally, the method 300 may include determining a fraction of the TDI that was delivered as boluses or “a total daily bolus dose,” as shown in act 304 of FIG. 3. For example, the method 300 may include determining a fraction (e.g., how much) of the TDI that was delivered responsive to user inputs and/or user instruction to deliver insulin via any of the manners described above in regard to FIG. 1. For instance, the total daily bolus dose may include insulin delivered responsive to a user input defining a bolus amount and/or details related to a meal consumed or about to be consumed.

In some embodiments, the total daily bolus dose may include only insulin delivered via the automated medicament delivery device 114 responsive to user inputs. In additional embodiments, the total daily bolus dose may also include insulin that was delivered via an insulin pen, injection, breath inhaler, or other manual delivery system. For instance, information regarding bolus doses delivered by an instrument or system other than the automated medicament delivery device 114 may be input by the user and/or tracked by the handheld electronic computing device 138 or other portion of the system 100, and the handheld electronic computing device 138 may provide the information to the medicament delivery system 200. As a result, those bolus doses delivered by the instrument or system other than the automated medicament delivery device 114 may be included in the total daily bolus dose. In some embodiments, the controller 208 of the medicament delivery system 200 may determine the percentage of the TDI that was delivered within the total daily bolus dose. As a non-limiting example, the controller 208 of the medicament delivery system 200 may determine the fraction by dividing the total daily bolus dose by the TDI. The determined fraction is referred to hereinafter as the “actual bolus fraction.”

The method 300 may further include determining a ratio of the actual bolus fraction relative to a target bolus fraction, as shown in act 306 of FIG. 3. For example, the controller 208 of the medicament delivery system 200 may determine the ratio of the actual bolus fraction relative to a target bolus fraction. As noted above, the target bolus fraction may include a particular fraction (e.g., percentage) of a TDI that has been determined based on clinical studies and clinical data. For instance, as an example, the target or “ideal” bolus fraction may be about 0.5 or 50%. Therefore, utilizing the foregoing target bolus fraction within the foregoing example, boluses (e.g., manual insulin delivery) should account for about 50% of a user's TDI.

As a non-limiting example, the actual bolus fraction of a given user within a given 24-hr period may be 0.3 and the target bolus fraction may be 0.5; accordingly, a ratio of the actual bolus fraction to an exemplary target or “ideal” bolus fraction may be

$$\frac{0.3}{0.5}$$

or 60%. Accordingly, the amount of insulin delivered via the total daily bolus dose in the above example was only 60% of the target daily bolus dose. For purposes of the present disclosure and for clarity, the target bolus fraction is shown as being 0.5 or 50%; however, the disclosure is not so limited; and one of ordinary skill in the art will readily recognize that bolus targets and the target fraction of boluses of a TDI may vary from one user to another and may be dependent on the user's insulin delivery habits and the medicament delivery device. As such, other values for the target bolus fraction fall within the scope of the present disclosure.

Additionally, the method 300 may include, based at least partially on the determined ratio, determining one or more new values of parameters to be recommended to a user for change and/or be utilized by the automated medicament delivery device 114 to calculate bolus doses (e.g., manual insulin delivery), as shown in act 308 of FIG. 3. For example, the controller 208 of the medicament delivery system 200 may determine the one or more new values of parameters (e.g., clinical parameters) to be recommended to a user for change and/or be utilized by the automated medicament delivery device 114 to calculate bolus doses (e.g., manual insulin delivery). In some embodiments, the one or more new values of parameters may include a new insulin to carbohydrate ratio (new IC ratio) and/or a new correction factor (new CF). For instance, the one or more new values of parameters may include adjusted parameters (e.g., new parameter values (e.g., new values for parameters such as the IC ratio and/or the CF)). In some embodiments, one or more of the one or more new values of parameters may be used only to calculate bolus doses and not used to calculate basal or automated doses of medicament delivery. Hence, in some embodiments, there may be at least two separate IC ratios and at least two separate CF values that may be utilized in calculations, one for bolus calculations and one for basal calculations.

As non-limiting examples, the new IC ratio and the new CF may be determined based on the determined ratio according to the following Equations 2 and 3:

$${IC}_{new} ( i ) = IC \frac{( \frac{{\sum }_{k = 1}^{288} {I}_{m} ( {j}_{i} - k )}{TDI ( i )} )}{0.5} \qquad Equation 2$$

$${CF}_{new} ( i ) = CF \frac{( \frac{{\sum }_{k = 1}^{288} {I}_{m} ( {j}_{i} - k )}{TDI ( i )} )}{0.5} \qquad Equation 3$$

where IC_{new}(i) is the new IC ratio, CF_{new} (i) is the new CF, IC is a previous IC ratio previously input by a user and/or provider, stored in memory, and previously utilized to calculate bolus doses, CF is a previous CF previously input by a user and/or provider, stored in memory, and previously utilized to calculate bolus doses,

$${\sum }_{k = 1}^{288} {I}_{m} ( {j}_{i} - k )$$

is the sum of boluses (Iₘ) (i.e., manual insulin doses) delivered during a time period of, for example two-hundred eighty-eight (288) cycles of 5-minute increments, or 24 hours (i.e., the total daily bolus dose), jᵢ represents the manual insulin doses for a current cycle, and k represents the manual insulin doses for the previous two-hundred eighty-eight (288) cycles. Furthermore, while Equations 2 and 3 show a time period of two-hundred eighty-eight (288) cycles or 24 hours, the disclosure is not so limited, and any time period and/or number of cycles can be utilized. Further, instead of the CF or IC values, which may have been previously input by a user and/or provider and stored in memory, any other sources of CF or IC values may also be utilized, such as a combination of the user's input values and other values calculated from other sources.

As a non-limiting example, if the user's actual bolus fraction is 0.3 (i.e., 30%) instead of the target bolus fraction of 0.5 (i.e., 50%), the previous IC ratio may be multiplied by the determined ratio

$$( i . e . , \frac{0.3}{0.5} or 60 \% ) ,$$

yielding a new IC ratio that is 40% less than the previous IC ratio, according to exemplary Equation 2 above. In other words, the previous IC ratio is decreased by 40% to arrive at the new IC ratio in this example.

Furthermore, as is known in the art, the IC ratio represents how many grams of carbohydrates are calculated to be addressed (e.g., disposed of or metabolized) by one unit of insulin, e.g., rapid-acting insulin. Accordingly, decreasing the IC ratio (e.g., decreasing the number of grams of carbohydrates that are calculated to be addressed (e.g., disposed of or metabolized) by one unit of rapid-acting insulin within an algorithm determining a bolus amount for manual insulin delivery), increases the number of units to be delivered to address a given carbohydrate amount of a consumed or to be consumed meal. As a result, subsequent to the determination of the new IC ratio, should the new IC ratio be implemented in calculating bolus doses, when the user makes a bolus request (e.g., a request for manual insulin delivery responsive to meal consumption), an amount of insulin delivered in the bolus (i.e., manual insulin delivery) will be increased, and a total daily bolus dose for the respective day will be increased, which will cause the actual bolus fraction to more closely match (e.g., approach) the target bolus fraction of the TDI. Likewise, increasing the IC ratio will decrease amounts of insulin delivered in subsequent bolus doses. As mentioned above, in exemplary embodiments the new IC ratio may be used only for determining bolus doses, and not used for determining basal doses.

Additionally, the new CF may be determined in the same manner as the new IC ratio according to the above exemplary equations. Similar to the IC ratio, decreasing the CF (i.e., implementing the new CF) will increase an amount of insulin delivered in subsequent boluses (i.e., manual insulin deliveries) and vice-versa. And as mentioned above, in exemplary embodiments the new CF may be used only for determining bolus doses, and not used for determining basal doses.

As is known in the art, a bolus dose may be determined utilizing the Equation 4:

$$Bolus dose = \frac{Total grams of carbohydrates in a meal}{IC ratio} + \frac{Current blood glucose - Target blood glucose}{CF} \qquad Equation 4$$

Accordingly, as a non-limiting example, if both the new IC ratio and the new CF represent decreases of the parameters by 40%, the total daily bolus dose for a day subsequent to implementing the new IC ratio and the new CF will be increased by 66.6%.

Referring still to FIG. 3, in some embodiments, responsive to determining the new IC ratio and the new CF ratio, the method 300 may include providing a recommendation to the user or a provider to change one or more parameters for calculating bolus doses based on the determined one or more new values of parameters, as shown in act 312 of FIG. 3. For example, the controller 208 of the medicament delivery system 200 may cause a recommendation that the one or more parameters be changed to the determined one or more new values of parameters. In some embodiments, the controller 208 may cause the recommendation to be displayed on the user interface 148 of the handheld electronic computing device 138. In additional embodiments, the controller 208 may cause the recommendation to be displayed on a user interface of the automated medicament delivery device 114 and/or the medicament delivery system 200 and/or another device such as a smartwatch or other display. In some embodiments, providing the recommendation may include providing the recommendation to one or more other devices. For instance, the recommendation may be provided to a manual insulin delivery system (e.g., an insulin pen), and the manual insulin delivery system may utilize the recommended one or more new values of parameters to calculate subsequent bolus doses. In some embodiments, the new values of parameters may be changed without requiring user acceptance of the new values of parameters and/or without displaying the new parameter values in, for example, a pop-up window for the user's immediate review.

In some embodiments, the one or more new values of parameters (i.e., the new IC ratio and the new CF) may be optionally adjusted (e.g., rate-adjusted) prior to making a recommendation to a user to change the parameters and/or utilizing the new values of parameters to calculate bolus doses (e.g., manual insulin delivery), as shown in act 310 of FIG. 3. For instance, the new values of parameters may be adjusted based on a selected rate of adaptation that can be selected/determined based at least partially on an availability of new data. For instance, when new data is available relatively often, a smaller rate of adaptation (e.g., 0.1 or 0.2) may be selected, and when new data is available only intermittently, a larger rate of adaptation (e.g., 0.6 or 0.7 or more) may be selected.

The new IC ratio and the new CF may be rate-adjusted utilizing algorithms implementing a rate of adaptation and considering both the previous IC ratio and CF and the new IC ratio and CF determined above in Equations 2 and 3. Furthermore, the algorithms may weigh the previous IC ratio and CF differently than the new IC ratio and CF. For example, the algorithms may give less weight (e.g., 20%) to the new IC ratio and CF than to the previous IC ratio and CF (e.g., 80%), thus lessening an amount by which the parameters are adapted or recommended to be changed responsive to a single iteration of the method 300 (i.e., responsive to only 24 hours worth of data). As a result, the IC ratio and CF may be iteratively adjusted over time as a moving average of the previous parameters and the new values of parameters at a selectable rate of adaptation. And as mentioned above, the exemplary methods discussed above and below may be performed repeatedly and new parameter values may be calculated repeatedly, such as every day, or every cycle, or every N cycles or N days, for example.

As a non-limiting example, the new IC ratio and the new CF may be adjusted utilizing Equations 5 and 6 below:

$${IC}_{f} ( i ) = ( 1 - 0.2 {T}_{new} ) {IC}_{f} ( i - 1 ) + 0.2 {T}_{new} {IC}_{new} ( i ) \qquad Equation 5$$

$${CF}_{f} ( i ) = ( 1 - 0.2 {T}_{new} ) {CF}_{f} ( i - 1 ) + 0.2 {T}_{new} {CF}_{new} ( i ) \qquad Equation 6$$

where 0.2 is an exemplary selected rate of adaptation, IC_{ƒ}(i) and CF_{ƒ}(i) represent the rate-adjusted new IC ratio and the rate-adjusted new CF, IC_{ƒ} and CF_{ƒ} represent the previous IC ratio and CF, and T_{new} represents the time period after which the new value of the parameter is recalculated (how often the calculation is performed). For instance, T_{new} may be equal to an hour, six (6) hours, twelve (12) hours, twenty-four (24) hours, forty-eight (48) hours, seventy-two (72) hours, etc. Furthermore, as a result, T_{new} represents a period of time for which new data (e.g., measurements and dosing data) is available. As is apparent from Equations 5 and 6, the rate of adaptation can be selected (e.g., tailored or tuned) to give more or less weight to the previous IC ratio and CF and to conversely give less or more weight to the new IC ratio and CF. As a result, a rate at which the parameters are changed based on new data can be selected (e.g., tuned).

As a non-limiting example, if a total daily bolus dose during a previous two-hundred eighty-eight (288) cycles for a user was 10 units of insulin, and the user's TDI was 24 units of insulin, the Equations 2 and 3 would provide a new IC ratio and a new CF that are decreased by 18% decrease relative to a previous IC ratio and previous CF. However, Equations 5 and 6 would decrease the effect the new IC ratio and a new CF would have on an actually recommended new IC ratio and CF. Accordingly, as noted above, the IC ratio and CF may be iteratively adjusted over time as a moving average of the previous parameters and the new values of the parameters at a selectable rate of adaptation.

Responsive to adjusting the new IC ratio and the new CF, the method 300 may include proceeding to act 312 and providing a recommendation to the user to change to the one or more parameters based on the determined one or more new values of the parameters according to any of the manners described above.

Referring still to act 312, in some embodiments, the method 300 may require user input to change the parameters. For example, the user may have to manually change the parameters within the memory 120 of the automated medicament delivery device 114 (e.g., within settings of the automated medicament delivery device 114), or the user may have to manually change the parameters in another application on, for example, the handheld electronic computing device 138 or on another device. In additional embodiments, the controller 208 of the medicament delivery system 200 may change the parameters with the memory 120 of the automated medicament delivery device 114 responsive to a user input indicating acceptance of the recommendation by the user. In some embodiments, the parameters may be automatically adjusted by the controller 208 of the medicament delivery system 200 without user input. For example, the controller 208 may adjust the parameters stored in memory and utilized to calculate bolus doses.

Referring still to FIG. 3, subsequent to the previous one or more parameters being changed to the one or more new values of the parameters, the method 300 may further include generating at least one request 214 to administer a bolus dose according to the one or more new values of the parameters, as shown in act 314 of FIG. 3. For example, subsequent to a user manually changing one or more previous parameters to the recommended one or more new values of the parameters, or the one or more previous parameters being automatically changed to the one or more new values of the parameters, the controller 208 may generate at least one request 214 to administer a bolus dose based on the one or more new values of the parameters. For instance, responsive to a user input indicating a consumed meal or a meal to be consumed, the controller 208 may generate at least one request 214 to administer a bolus dose based on the one or more new values of the parameters and according to any of the manners described above in regard to FIG. 1 and FIG. 2.

Additionally, the method 300 may include providing the generated at least one request to the automated medicament delivery device 114, as shown in act 316 of FIG. 3. For instance, the controller 208 may provide the at least one request to the delivery mechanism controller 210 of the delivery system 202 of the medicament delivery system 200 via any of the manners described above in regard to FIG. 1 and FIG. 2.

Moreover, the method 300 may optionally include causing the medicament delivery system 200 to deliver insulin to the user's body responsive to the request 214 to administer a dose, as shown in act 318 of FIG. 3. For instance, the method 300 may include causing the medicament delivery system 200 to deliver insulin to the user's body responsive to the request 214 to administer a dose via any of the manners described above in regard to FIG. 1 and FIG. 2.

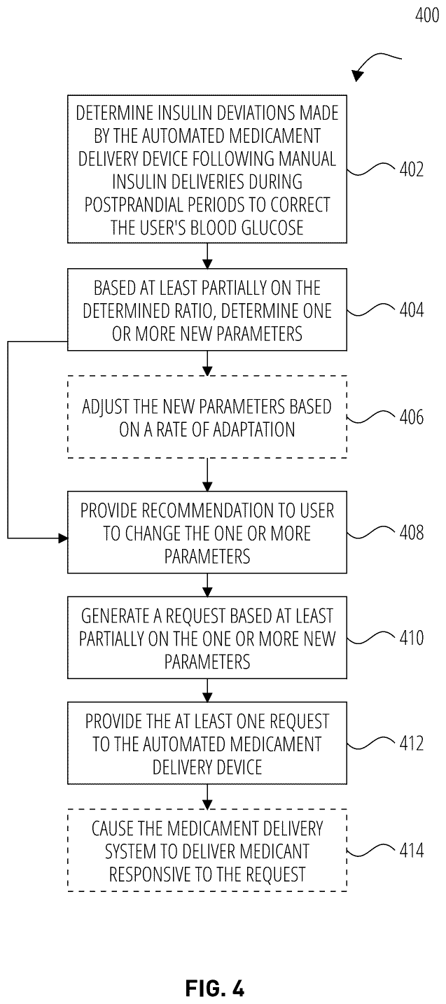

FIG. 4 is a flowchart of a method 400 of recommending new values of the parameters utilized to calculate manual insulin doses (i.e., bolus doses) according to one or more embodiments of the present disclosure. In particular, the method 400 may include recommending new clinical parameters utilized to calculate bolus doses based at least partially on excursions of insulin delivery (e.g., suspensions of insulin delivery and/or additional insulin deliveries) executed by the AID algorithm following each manual insulin delivery (i.e., bolus) to correct a user's blood glucose. For purposes of the present disclosure, the excursions are referred to herein as insulin deviations, and the quantity of insulin (e.g., the amount of additional insulin delivered or the lack of insulin deliveries) represented in these insulin deviations may be utilized to recommend new values of the parameters utilized by the automated medicament delivery device 114 when determining and administering manual insulin doses.

The method 400 may include determining insulin deviations made by the automated medicament delivery device 114 following bolus doses during postprandial periods to correct the user's blood glucose levels, as shown in act 402 of FIG. 4. For example, the controller 208 of the medicament delivery system 200 may determine insulin deviations made by the automated medicament delivery device 114 following bolus doses during postprandial periods to correct the user's blood glucose levels.

In some embodiments, the insulin deviations (I_{dev,n}) may be determined via the following Equation 7:

$${I}_{dev , n} = {\sum }_{k = 1}^{60} {I}_{AID} ( {j}_{n} + k ) - \frac{T D I}{48} \cdot \frac{1}{12} \qquad Equation 7$$

where the insulin delivered during each cycle of a postprandial period (in this example, 60 cycles and 5 hours) after each bolus is compared against an expected hourly insulin need and/or expected TDI-based basal in each cycle to determine the insulin deviations (I_{dev,n}). While the foregoing example is given with a postprandial period of 60 cycles and 5 hours, the disclosure is not so limited, and any conventional postprandial period may be utilized.

Additionally, the method 400 may include, based at least partially on the determined insulin deviations, determining one or more new values of the parameters to be recommended to a user for change and/or be utilized by the automated medicament delivery device 114 to calculate bolus doses (e.g., manual insulin delivery), as shown in act 404 of FIG. 4. For example, the controller 208 of the medicament delivery system 200 may determine one or more new values of the parameters to be recommended to a user for change and/or be utilized by the automated medicament delivery device 114 to calculate bolus doses. In some embodiments, the one or more parameters may include the IC ratio and/or the CF. In one or more embodiments, the one or more new values of the parameters may be determined utilizing the following Equations 8 and 9:

$${IC}_{new} ( i ) = I C \frac{{\sum }_{k = 1}^{N} {I}_{manual , k}}{{\sum }_{k = 1}^{N} {I}_{dev , k} + {I}_{manual , k}} \qquad Equation 8$$

$${CF}_{new} ( i ) = CF \frac{{\sum }_{k = 1}^{N} {I}_{manual , k}}{{\sum }_{k = 1}^{N} {I}_{dev , k} + {I}_{manual , k}} \qquad Equation 9$$

where a total additional insulin deviation of N boluses with a current i^{th} automated medicament delivery device 114 cycle (e.g., pump cycle) is compared to the total manual insulin delivery within the current i^{th} automated medicament delivery device 114 cycle (e.g., pump cycle). Furthermore, in some embodiments, the parameters may be adjusted over time as a moving average of previous parameters and adjusted parameters at a tunable rate.

Referring still to FIG. 4, in some embodiments, responsive to determining the new IC ratio and the new CF ratio, the method 400 may include providing a recommendation to the user or a provider to change one or more parameters for calculating bolus doses based on the determined one or more new values of the parameters, as shown in act 408 of FIG. 4. For example, the controller 208 of the medicament delivery system 200 may cause a recommendation that the one or more parameters be changed to the determined one or more new values of the parameters. In some embodiments, the controller 208 may cause the recommendation to be displayed on the user interface 148 of the handheld electronic computing device 138. In additional embodiments, the controller 208 may cause the recommendation to be displayed on a user interface of the automated medicament delivery device 114 and/or the medicament delivery system 200 and/or another device such as a smartwatch or other display. In some embodiments, the new values of the parameters may be changed without requiring user acceptance of the new values of the parameters and/or without displaying the new parameter values in, for example, a pop-up window for the user's immediate review.

In some embodiments, the one or more new values of the parameters (i.e., the new IC ratio and the new CF) may be optionally adjusted (e.g., rate-adjusted) prior to making a recommendation to a user to change the parameters and/or utilizing the new values of the parameters to calculate bolus doses (e.g., manual insulin delivery), as shown in act 406 of FIG. 4. For instance, the new values of the parameters may be adjusted via any of the manners described above in regard to act 310 of FIG. 3.

Referring again to act 408, in some embodiments, the method 400 may require user input to change the parameters. For example, the user may have to manually change the parameters within the memory 120 of the automated medicament delivery device 114 (e.g., within settings of the automated medicament delivery device 114), or the user may have to manually change the parameters in another application on, for example, the handheld electronic computing device 138 or on another device. In additional embodiments, the controller 208 of the medicament delivery system 200 may change the parameters with the memory 120 of the automated medicament delivery device 114 responsive to a user input indicating acceptance of the recommendation by the user. In some embodiments, the parameters may be automatically adjusted by the controller 208 of the medicament delivery system 200 without user input. For example, the controller 208 may adjust the parameters stored in memory and utilized to calculate bolus doses.

Subsequent to the previous one or more parameters being changed to the one or more new values of the parameters, the method 400 may further include generating at least one request 214 to administer a bolus dose according to the one or more new values of the parameters, as shown in act 410 of FIG. 4. For example, subsequent to a user manually changing one or more previous parameters to the recommended one or more new values of the parameters or the one or more previous parameters being automatically changed to the one or more new values of the parameters, the controller 208 may generate at least one request 214 to administer a bolus dose based on the one or more new values of the parameters. For instance, responsive to a user input indicating a consumed meal or a meal to be consumed, the controller 208 may generate at least one request 214 to administer a bolus dose based on the one or more new values of the parameters and according to any of the manners described above in regard to FIG. 1 and FIG. 2. And as mentioned above, in some embodiments, one or more of the one or more new values of the parameters, or newly adjusted parameters, may be used only to calculate bolus doses and not used to calculate basal or automated doses of medicament delivery.

Additionally, the method 400 may include providing the generated at least one request to the automated medicament delivery device 114, as shown in act 412 of FIG. 4. For instance, the controller 208 may provide the at least one request to the delivery mechanism controller 210 of the delivery system 202 of the medicament delivery system 200 via any of the manners described above in regard to FIG. 1 and FIG. 2.

Moreover, the method 400 may also optionally include causing the medicament delivery system 200 to deliver insulin to the user's body responsive to the request 214 to administer a dose, as shown in act 414 of FIG. 4. For instance, the method 400 may include causing the medicament delivery system 200 to deliver insulin to the user's body responsive to the request 214 to administer a dose via any of the manners described above in regard to FIG. 1 and FIG. 2.

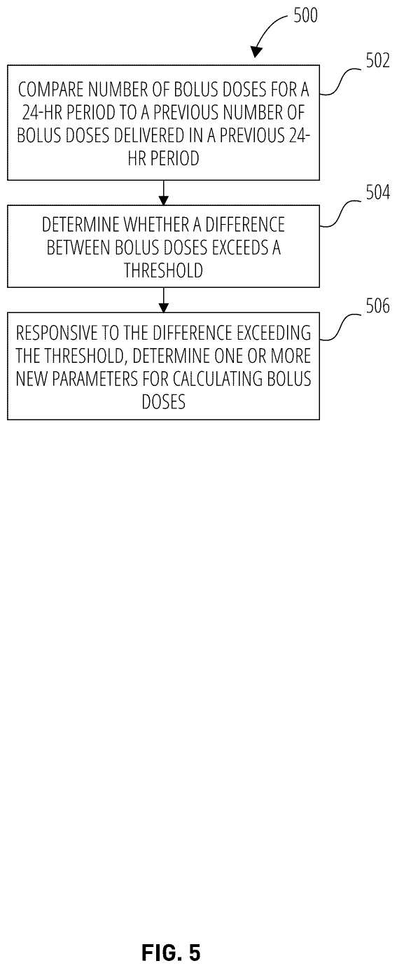

FIG. 5 is a flowchart of a method 500 of recommending new values of the parameters (or adjusting old parameters stored in memory) utilized to calculate manual insulin doses (i.e., bolus doses) according to one or more embodiments of the present disclosure. In particular, the method 500 may include recommending new clinical parameters utilized to calculate bolus doses based at least partially on a change in a number of boluses being delivered by a user during a period of time.

The method 500 may include comparing a recent number of bolus doses delivered within a period of time to a reference number of bolus doses, as shown in act 502 of FIG. 5. For example, the controller 208 of the medicament delivery system 200 may compare a recent number of bolus doses delivered within a 24-hr period to the reference number of bolus doses. In some embodiments, the 24-hr period may be a most-recent 24-hr period (e.g., a rolling 24-hr period). As a non-limiting example, the controllers 208 may query one or more memories to compare the recent number of bolus doses delivered within a 24-hr period to the reference number of bolus doses.

In some embodiments, the reference number of bolus doses includes a number of bolus doses delivered during a previous period of time (e.g., a previous 24-hr period of time). In some embodiments, the reference number of bolus doses may include a number of bolus doses delivered within a 24-hr period immediately preceding the most-recent 24-hr period. In some embodiments, the reference number of bolus doses may include a multiple day (e.g., 2-day, 3-day, 7-day) average of bolus doses delivered.

Additionally, the method 500 may include determining whether a difference between the recent number of boluses delivered and the reference number of bolus doses exceeds a threshold, as shown in act 504 of FIG. 5. For instance, the controller 208 of the medicament delivery system 200 may determine whether a difference between the recent number of bolus doses and the reference number of bolus doses exceeds a threshold.

In some embodiments, the threshold may include a percentage change. For example, the threshold may include a percentage of the reference number of bolus doses. As non-limiting examples, the threshold may include 10%, 20%, 30%, 50%, or any other percentage of the reference number of bolus doses. In additional embodiments, the threshold may include a whole number. For instance, the threshold may include any number of one (1) through ten (10) or more.

In one or more embodiments, the threshold may be stored within one or more memories of the system 100 and may be selectable by a user and/or a provider. In some embodiments, the threshold is determined based on clinical recommendations, studies, and/or data.

Responsive to a determination that the difference between the recent number of bolus doses and the reference number of bolus doses exceeds the threshold, the method 500 may include determining one or more new values of the parameters utilized in determining bolus doses, as shown in act 506 of FIG. 5. For instance, in some embodiments, the controller 208 of the medicament delivery system 200 may determine one or more new values of the parameters. In some embodiments, the one or more new values of the parameters (or newly adjusted parameters) may include a new (or adjusted) insulin to carbohydrate ratio (new IC ratio) and/or a new correction factor (new CF).

In some embodiments, the new IC ratio and the new CF may be determined based on the difference between the recent number of bolus doses and the reference number of bolus doses. For instance, if the difference is a decrease of 25% or the recent number of bolus doses is ¾ of the reference number of bolus doses, the IC ratio and the CF may also be decreased by 25% or multiplied by ¾ to yield adjusted or a new IC ratio and CF. Specifically, in place of, or in addition to, considering an amount of insulin delivered manually, a frequency of manual delivery may be utilized in each of the embodiments described herein. As a non-limiting example, an automated insulin delivery device may determine that the system may perform more optimally if a user provides an average of five manual insulin deliveries over a period of time such as one day. Thus, if the current user typically provides an average of three manual insulin deliveries per day, the user's manual insulin delivery parameters may be decreased by a factor, such as 40% (or 1−(⅗)), which may improve overall system performance.

Furthermore, as is known in the art, the IC ratio represents how many grams of carbohydrates are calculated to be addressed (e.g., disposed of or metabolized) by one unit of rapid-acting insulin. Accordingly, decreasing the IC ratio (e.g., decreasing the number of grams of carbohydrates that are calculated to be addressed (e.g., disposed of or metabolized) by one unit of rapid-acting insulin within an algorithm determining a bolus amount for manual insulin delivery), increases the number of units to be delivered to address a given carbohydrate amount of a consumed or to be consumed meal. As a result, subsequent to the determination of the new IC ratio, should the new IC ratio be implemented in calculating bolus doses, when the user makes a bolus request (e.g., a request for manual insulin delivery responsive to meal consumption), an amount of insulin delivered in the bolus (i.e., manual insulin delivery) will be increased, and a total daily bolus dose for the respective day will be increased, which will cause the actual bolus fraction to more closely match (e.g., approach) the target bolus fraction of the TDI. Likewise, increasing the IC ratio will decrease amounts of insulin delivered in subsequent bolus doses.

Additionally, the method 500 may include any of acts 312-318 described above in regard to FIG. 3.

Referring to FIG. 1 through FIG. 5 together, the methods and systems described herein provide advantages over conventional methods and systems for maintaining a patient's blood glucose level within a desired range. An automated medicament delivery device and a control algorithm governing one or more operations of the automated medicament delivery device may typically optimized (e.g., tuned) based on an assumption that 50% of a user's insulin needs are fulfilled by manual insulin delivery responsive to the user eating meals (i.e., bolus doses). As a result, by adjusting one or more parameters utilized in calculating bolus doses and bringing the user's actual bolus patterns into alignment with assumptions of a control algorithm and an automated medicament delivery device, the methods and systems of the present disclosure may improve effectiveness' of control algorithms and associated systems (e.g., automated medicament delivery device) in managing blood glucose levels of users.

In the detailed description, reference is made to the accompanying drawings, which form a part hereof, and in which are shown, by way of illustration, specific examples of examples in which the present disclosure may be practiced. These examples are described in sufficient detail to enable a person of ordinary skill in the art to practice the present disclosure. However, other examples may be utilized, and structural, material, and process changes may be made without departing from the scope of the disclosure.

The illustrations presented herein are not meant to be actual views of any particular method, system, device, or structure, but are merely idealized representations that are employed to describe the examples of the present disclosure. The drawings presented herein are not necessarily drawn to scale. Similar structures or components in the various drawings may retain the same or similar numbering for the convenience of the reader; however, the similarity in numbering does not mean that the structures or components are necessarily identical in size, composition, configuration, or any other property.

The description may include examples to help enable one of ordinary skill in the art to practice the disclosed examples. The use of the terms “exemplary,” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

It will be readily understood that the components of the examples as generally described herein and illustrated in the drawing could be arranged and designed in a wide variety of different configurations. Thus, the description of various examples is not intended to limit the scope of the present disclosure, but is merely representative of various examples. While the various aspects of the examples may be presented in drawings, the drawings are not necessarily drawn to scale unless specifically indicated.

Furthermore, specific implementations shown and described are only examples and should not be construed as the only way to implement the present disclosure unless specified otherwise herein. Elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

In the Brief Summary and in the Detailed Description, the claims, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and examples described herein.

Those of ordinary skill in the art would understand that information and signals may be represented using any of a variety of different technologies and techniques. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative methods, logical blocks, modules, and circuits described in connection with the examples of the system 100, and in particular, the automated medicament delivery device 114 and the handheld electronic computing device 138, disclosed herein may be implemented or performed with a general purpose processor, a special purpose processor, a digital signal processor (DSP), an Integrated Circuit (IC), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor (may also be referred to herein as a host processor or simply a host) may be a microprocessor, but in the alternative, the processor may be any conventional processor, controller, microcontroller, or state machine. A processor may also be implemented as a combination of computing devices, such as a combination of a DSP and a microprocessor, a plurality of microprocessors, one or more microprocessors in conjunction with a DSP core, or any other such configuration. A general-purpose computer including a processor is considered a special-purpose computer while the general-purpose computer is configured to execute computing instructions (e.g., software code) related to examples of the present disclosure.

The examples may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

All references cited herein are incorporated herein in their entireties. If there is a conflict between definitions herein and in an incorporated reference, the definition herein shall control.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternate useful combinations of the elements described, will become apparent to those skilled in the art from the description. Such modifications and embodiments also fall within the scope of the appended claims and equivalents.

What is claimed is:

1. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
determine an actual bolus fraction of a total daily insulin that was delivered as a total daily bolus dose;
determine a ratio of the actual bolus fraction relative to a target bolus fraction; and
based at least partially on the determined ratio of the actual bolus fraction relative to a target bolus fraction, determine at least one new value of a parameter utilized by the automated medicament delivery device to determine bolus doses.

2. The system of claim 1, wherein the parameter comprises at least one of an insulin to carbohydrate ratio or a correction factor.

3. The system of claim 1, comprising instructions that, when executed by the at least one processor, cause the automated medicament delivery device to automatically change at least one previously utilized value of the parameter to the at least one new value of the parameter without user input.

4. The system of claim 1, comprising instructions that, when executed by the at least one processor, cause the automated medicament delivery device to:
provide a recommendation to change at least one previously utilized value of the parameter to the at least one new value of the parameter; and
responsive to a user input, change the at least one previously utilized value of the parameter to the at least one new value of the parameter.

5. The system for administration of claim 4, comprising instructions that, when executed by the at least one processor, cause the automated medicament delivery device to generate a request to deliver a bolus dose to the user based at least partially on the at least one new value of the parameter.

6. The system of claim 5, comprising instructions that, when executed by the at least one processor, cause the automated medicament delivery device to deliver the bolus dose responsive to the generated request.

7. The system of claim 1, wherein the target bolus fraction is about 0.5.

8. The system of claim 1, wherein the parameter comprises an insulin to carbohydrate ratio, and wherein the at least one new value of the parameter is determined via the equation:
$${IC}_{new} ( i ) = IC \frac{( \frac{{\sum }_{k = 1}^{288} {I}_{m} ( {j}_{i} - k )}{TDI ( i )} )}{0.5}$$
where:
i is an index identifying the current adjustment cycle,
IC_{new} is the updated insulin-to-carbohydrate ratio computed at the iₜₕ adjustment cycle,
IC is the insulin-to-carbohydrate ratio stored immediately prior to the iₜₕ adjustment cycle,
k is an index identifying a current dosing interval within a 24-hour period,
jᵢ is a dosing-interval index at which the iₜₕ adjustment cycle is performed,
Iₘ(jᵢ−k) is the amount of insulin delivered manually by the insulin delivery device during the dosing interval indexed by (jᵢ−k),
$${\sum }_{k = 1}^{288} {I}_{m} ( {j}_{i} - k )$$
is the total manual insulin delivered over the most recent 288 dosing intervals,
TD(i) is the total daily insulin delivered (including both automated and manual deliveries) during the monitoring period ending at the iₜₕ adjustment cycle, and
0.5 is a fixed normalization constant corresponding to a 50% target proportion of manual insulin delivery relative to total daily insulin.

9. The system of claim 1, wherein the parameter comprises a correction factor, and wherein the at least one new value of the parameter is determined via the equation:
$${CF}_{new} ( i ) = CF \frac{( \frac{{\sum }_{k = 1}^{288} {I}_{m} ( {j}_{i} - k )}{TDI ( i )} )}{0.5} ,$$
where:
i is an index identifying the current adjustment cycle,
IC_{new} is the updated insulin-to-carbohydrate ratio computed at the iₜₕ adjustment cycle,
IC is the insulin-to-carbohydrate ratio stored immediately prior to the iₜₕ adjustment cycle,
k is an index identifying a current dosing interval within a 24-hour period,
jᵢ is a dosing-interval index at which the iₜₕ adjustment cycle is performed,
Iₘ(jᵢ−k) is the amount of insulin delivered manually by the insulin delivery device during the dosing interval indexed by (jᵢ−k),
$${\sum }_{k = 1}^{288} {I}_{m} ( {j}_{i} - k )$$
is the total manual insulin delivered over the most recent 288 dosing intervals,
TD(i) is the total daily insulin delivered (including both automated and manual deliveries) during the monitoring period ending at the iₜₕ adjustment cycle, and
0.5 is a fixed normalization constant corresponding to a 50% target proportion of manual insulin delivery relative to total daily insulin.

10. The system of claim 1, comprising instructions that, when executed by the at least one processor, cause the automated medicament delivery device to adjust the at least one new value of the parameter based at least partially on a selected rate of adaptation prior to providing a recommendation to change at least one previous utilized value of the parameter to the at least one new value of the parameter.

11. The system of claim 10, wherein adjusting the at least one new value of the parameter based at least partially on a selected rate of adaptation comprises weighting the previously utilized parameter higher than the determined at least one new value of the parameter.

12. The system of claim 11, comprising wherein adjusting the at least one new value of the parameter via the equation:
$${IC}_{f} ( i ) = ( 1 - 0.2 {T}_{new} ) {IC}_{f} ( i - 1 ) + 0.2 {T}_{new} {IC}_{new} ( i ) ,$$
where:
i is an index identifying the current adjustment cycle,
IC_{f}(i) is the filtered insulin-to-carbohydrate ratio used for dosing at the iₜₕ adjustment cycle.
IC_{f}(i−1) is the filtered insulin-to-carbohydrate ratio from the immediately preceding (i−1) adjustment cycle,
IC_{new}(i) is a newly calculated insulin-to-carbohydrate ratio at the iₜₕ adjustment cycle, and
T_{new} is a dimensionless, cycle-based weighting parameter that represents how rapidly the filter responds to newly computed ratios.

13. The system of claim 1, comprising instructions that, when executed by the at least one processor, cause the at least one processor to:
compare a count of manual bolus doses over a most-recent 24-hour period to a count of manual bolus doses over a further 24-hour period, the further 24-hour period preceding the most-recent 24-hour period; and
determine at least one new value of the parameter utilized to calculate bolus doses at least partially responsive to the comparison.

14. The system of claim 13, comprising instructions that, when executed by the at least one processor, cause the at least one processor to:
determine the at least one new value of the parameter utilized to calculate bolus doses at least partially responsive to a difference between the count of manual bolus doses over a most-recent 24-hour period and the count of manual bolus doses over the further 24-hour period exceeding a predetermined threshold.

15. A method for recommending new values of parameters for determining bolus doses, the method comprising:
determining an actual amount of a total daily insulin that was delivered as bolus doses;
determining a ratio of the actual amount relative to a target amount; and
based at least partially on the determined ratio, determining at least one new value of a parameter to utilize in calculating bolus doses.

16. The method of claim 15, wherein the parameter comprises at least one of an insulin to carbohydrate ratio or a correction factor.

17. The method of claim 15, comprising automatically changing at least one previously utilized value of the parameter to the at least one new value of the parameter without user input.

18. The method of claim 15, comprising:
providing a recommendation to change at least one previously utilized value of the parameter to the at least one new value of the parameter; and
responsive to a user input, changing the at least one previously utilized value of the parameter to the at least one new value of the parameter.

19. The method of claim 18, further comprising, based at least partially on the determined at least one new value of the parameter, generating a request to deliver a bolus dose to a user.

20. The method of claim 19, further comprising, responsive to the generated request, delivering the bolus dose.

21. The method of claim 15, comprising:
comparing a count of manual bolus doses over a most-recent 24-hour period to a count of manual bolus doses over a further 24-hour period, the further 24-hour period preceding the most-recent 24-hour period; and
determining at least one new value of the parameter utilized to calculate bolus doses at least partially responsive to the comparing.

22. The method of claim 21, wherein the comparing comprises:
determine whether a difference between the count of manual bolus doses over a most-recent 24-hour period and the count of manual bolus doses over the further 24-hour period exceeding a predetermined threshold.

23. A system for administration of medicament to a user-body, the system comprising:
an analyte sensor; and
an automated medicament delivery device comprising:
at least one processor; and
at least one non-transitory computer-readable storage medium storing instructions thereon that, when executed by the at least one processor, cause the automated medicament delivery device to:
determine insulin deviations made by the automated medicament delivery device subsequent to administration of bolus dose during postprandial periods throughout a given period of time; and
based at least partially on the determined insulin deviations, determine at least one new value of a parameter to utilize in determining bolus doses.

24. The system for administration of claim 21, wherein the parameter comprises at least one of an insulin to carbohydrate ratio or a correction factor.
